(19) United States
(12) Patent Application Publication
(10) Pub. No.: US 2006/0013878 A1
(43) Pub. Date: Jan. 19, 2006

(54) Process for forming a gel containing an ingredient therein

(76) Inventors: Fukuji Ebihara, Kobe (JP); Kiyoko Hirata, Ashiya (JP)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/184,618

(22) Filed: Jul. 19, 2005

Related U.S. Application Data
(60) Provisional application No. 60/589,049, filed on Jul. 19, 2004.
(60) Provisional application No. 60/651,780, filed on Feb. 10, 2005.

(51) Int. Cl. A61K 9/14

(52) U.S. Cl. 424/484

(57) ABSTRACT

A process for absorbing an ingredient into a gel item to form a gel has the steps of providing an ingredient and a liquid media, homogenizing the ingredient and the liquid media in a mixer to form a mixture, providing a gel item capable of absorbing the liquid media and absorbing the mixture into the gel item to form a gel. The ingredient is insoluble in the liquid media, and in the mixture form micelles suspended in the liquid media. The micelles contain the ingredient and have an average micelle diameter. As the gel item has an average pore size which is greater than or equal to the average micelle diameter, the micelles containing the ingredient will be absorbed into the gel item to form the gel.

### CROSS REFERENCE TO RELATED APPLICATION

This application claims the benefit of U.S. Provisional Application No. 60/589,049, filed Jul. 19, 2004 and U.S. Provisional Application No. 60/651780, filed Feb. 10, 2005.

### FIELD OF THE INVENTION

The present invention relates to processes for making a gel. Specifically, the present invention relates to processes for absorbing an ingredient into a gel.

### BACKGROUND OF THE INVENTION

Many products are formed of gels into which an ingredient has been absorbed. Examples of typical gels include those used in medicines, foods, air fresheners, plant and garden materials, hair care products, paper diapers, cooling pads to reduce fevers, deodorizers, etc. Ingredients in such gels may include dyes, medicinal active agents, perfumes, flavorings, vitamins, minerals, etc. which are dissolved into a liquid media such as water or an oil, and then absorbed into the gel. While many of the gels and ingredients may be compatible with the liquid media, in some cases, the ingredient and the liquid media are either insoluble to sparingly soluble in each other. This can cause problems as micelles of the non-dominant phase (typically the ingredient) will form and such micelles may not easily absorb into the gel. In certain cases, the micelle will not absorb into the gel at all, and instead will merely coat the outside of the gel as the liquid media is absorbed. This in turn may lead to inefficient use of the ingredient, and/or a deterioration of the desired gel/ingredient properties.

Accordingly, the need exists for a gel which overcomes the limitations above, and a process for forming such a gel.

### SUMMARY OF THE INVENTION

The present invention relates to a process for absorbing an ingredient into a gel item to form a gel having the steps of providing an ingredient and a liquid media, homogenizing the ingredient and the liquid media in a mixer to form a mixture, providing a gel item capable of absorbing the liquid media and absorbing the mixture into the gel item to form a gel. The ingredient is insoluble in the liquid media, and in the mixture form micelles suspended in the liquid media. The micelles contain the ingredient and have an average micelle diameter. As the gel item has an average pore size which is greater than or equal to the average micelle diameter, the micelles containing the ingredient will be absorbed into the gel item to form the gel.

It has now been found that by coordinating the average micelle diameter and the average pore size of the gel item, a gel can be formed which contains the ingredient therein, rather than just on the outside. Furthermore, it has been found that such a gel possesses significant benefits over a gel where the ingredient is merely coated thereupon; for example, if the ingredient is a perfume, then a gel according to the present invention may provide a scent impression which accurately reflects the scent impression of the perfume itself as it was designed. The gel of the present invention more evenly distributes the ingredient throughout the gel, which may be important, for example to provide accurate time release of the ingredient to the surroundings. The gel of the present invention may also provide good absorbency of oils even with hydrophilic gels, improved storage stability, a more consistent and lasting perfume impact, a more controlled release of active ingredients over time, etc.

### DETAILED DESCRIPTION OF THE INVENTION

All temperatures herein are in degrees Celsius (° C.) unless otherwise indicated. As used herein, the term “comprising” means that other steps, ingredients, elements, etc. which do not adversely affect the end result can be added. This term encompasses the terms “consisting of” and “consisting essentially of”.

As used herein, the term “insoluble” indicates that the ingredient's solubility in the liquid media is less than 0.1% (w/w) and includes the term “sparingly soluble”.

The process herein is intended to facilitate absorption of an ingredient into a gel item to form a gel. The process is especially important where the ingredient is insoluble in the liquid media. In such cases, the ingredient will often form micelles suspended in the liquid media. Then, when the liquid media is absorbed into the gel item, the gel may act like a sieve or a semi-permeable membrane, and thereby sieving or “filtering out” the ingredient from the liquid media which has just been absorbed. This in turn results in a gel which contains a substantial amount of the liquid media and little, if any ingredient therein. In such a case, the ingredient, in effect is merely coated on the outside of the gel. As the entire purpose of forming the gel is to get the ingredient into the gel, this results in sub-optimal incorporation of the ingredient into the gel. This can also result in wasted ingredient, excess process steps, and/or the requirement that large excesses of ingredient be used to achieve satisfactory incorporation into the gel.

However, the present invention recognizes that the sieving effect above may be solved by coordinating the size of the micelle with the gel's pore size. This in turn, allows more efficient incorporation of the ingredient into the gel. Accordingly, the present invention relates to a process for absorbing an ingredient into a gel item to form a gel by providing an ingredient and a liquid media. The ingredient is typically selected from a perfume, a flavoring, a medicinal active, a biological active, a chemically active compound, a dye, a vitamin, a mineral, a pigment and a combination thereof. In an embodiment of the present invention, the ingredient is a perfume, a flavoring, a dye or a combination thereof. In another embodiment of the present invention, the ingredient is a perfume oil. In another embodiment herein, the ingredient is a chemically active compound, such as a polymer with reactive moieties thereupon. In an embodiment of the present invention, the chemically-active compound is a malodor removing active, preferably selected from the group consisting of a reactive polymer, a chlorine dioxide, a cyclodextrin, a titanium dioxide, a phtalocyanine, a zinc chloride, a copper compound, an iron compound, a reactive aldehyde, a plant extract, an activated carbon, a zeolite and a mixture thereof Such malodor removing actives are described in, for example, U.S. Provisional Patent Application No. 60/560795 to Nair, et al., filed on Apr. 8, 2004.

The liquid media is typically selected from water, an oil, an organic solvent, and a mixture thereof. In an embodiment of the present invention, the liquid media is water. Typically, the liquid media will be in great volumetric and weight excess as compared to the ingredient. In an embodiment of the invention, the liquid media is in greater than about 5 times volumetric excess of the ingredient. In another embodiment herein, the liquid media is of from about 8 times to about 1,000,000 times volumetric excess of the ingredient. In another embodiment herein, the liquid media is of from about 10 times to about 100 times volumetric excess of the ingredient. It is essential, however, that the ingredient and the liquid media be insoluble in each other, otherwise the above problem does not occur.

The ingredient and the liquid media are homogenized in a mixer to form a mixture which contains micelles, containing the ingredient, suspended within the liquid media. The mixer useful herein may be any device which combines the ingredient and the liquid media into a homogenized mixture. However, the mixer must be compatible with the liquid media and the ingredient. For example, if the ingredient is sensitive to shear, then a low shear mixer should be used. Conversely, if high shear is required in order to form a homogenized mixture from the ingredient and the liquid media, then a high shear mixer should be used. Thus, mixers useful herein include, for example kitchen blenders and mixers such as are used to prepare food, low shear dynamic mixers such as propeller mixers, disk mixers, turbine mixers, hydrofoil mixers, helix mixers, and anchor mixers; low shear static mixers, moderate speed mixers, high shear dynamic rotor stator mixers, etc. Examples of mixers useful herein include such commonly-available mixers such as the Y-tron series from Quadro, Milburn, N.J., USA; mixers from Loedige Gmbh, Paderborn and Mannheim Germany, mixers from IKA® Works, Inc. Wilmington, N.C., USA; mixers from Lightnin, Rochester N.Y., USA; mixers from Ekato Gmbh, Lorrach, Germany; Kemics mixers from Chemineer, Inc., Dayton, Ohio., USA; Koch Equipment LLC, Kansas City, Miss., USA; Sulzer Chemtech USA, Inc., Pasadena Tex., USA; Silverson Machines Inc., East Longmeadow, Mass., USA; and others. Mixers which reduce aeration and/or induce only low levels of added aeration during the mixing process may also be preferred in some instances.

The homogenized mixture will often contain micelles which may be barely visible or invisible to the naked eye. However, such micelles will have an average micelle diameter which can be measured by the test method described below.

A gel item is provided which is capable of absorbing the liquid media. The gel item may be a pre-formed gel which absorbs the liquid media via exchanging existing molecules supporting the gel structure with those of the liquid media. Alternatively, the gel item may be a gel precursor, such as a dehydrated gel, a powder, a chemical, a polymer, and/or a “gel chip”. A gel precursor therefore is not currently a gel, but contains the structure thereof or some chemicals which will react to form the gel, typically upon addition of the liquid media. The gel precursor then forms into a gel after absorbing, or because of absorbing the liquid media. Examples of the gel item useful herein include both natural or synthetic gels. Natural gels can be xanthan gum, guar gum, carboxy methyl cellulose or agars. Synthetic gel can be cross-linked polymers such as acrylic based polymers. The gel item can be chemically cross-linked or physically cross-linked. Examples of cross-linked polymers are cross-linked acrylic acid, acrylamide, polyethylene oxide, maleic acid, styrene, malic acid, etc., especially block polymers thereof. Examples of physically cross-linked polymers are polyethylene oxides. Examples of gel items useful herein includes Aquakeep, Aquacube, Aquacalk TW, and Aquacalk TWB from Sumitomo Seika, Osaka, Japan, Aquapearl from Mitsubishi Chemicals, Tokyo, Japan, and Aqualin, AQUALIC CA, AAULIC CS, ACRYHOPE, and super absorption polymer from Nihon Shyokubai, Osaka, Japan. In a preferred embodiment, the gel item is a gel precursor. In a preferred embodiment the liquid media is water and the gel item is a dehydrated gel. In a preferred embodiment the gel item is formed of a polymer, such as a block polymer.

The gel can be made by combining a dispersion medium such as water, solvent, a solution of active ingredients or mixture of ingredients with the disperse phase such as naturally occurring materials xanthum, agar, alginate, wood pulp, guar or synthetic absorbent polymer such as cross-linked or non cross-linked or partially cross-linked poly acrylic acid, poly acrylamide, poly(ethylene oxide), poly(vinyl alcohol), carboxy methyl cellulose (CMC) and the like. Many more such examples can be found in, for example, Modern Superabsorbent Polymer Technology (Wiley-VCH, 1997), Fredric L. Buchholz and Andrew T. Graham editors.

The gel item has an average pore size which is typically the size of the holes in the gel structure for a pre-formed gel, or the size of the holes in the gel structure which will be formed from a gel precursor. It is recognized that in the case where a pre-formed gel is used, and the liquid media is exchanged for the pre-existing molecules, the pore size may change significantly. For example, if a polar solvent within a pre-formed gel is exchanged for a non-polar solvent (as the liquid media), then the gel structure may change significantly in terms of the pore size, physical properties and/or molecular interactions. Thus, in such a case, the pore size is measured at the time the ingredient is to be absorbed, rather than before or afterwards. The pore size for certain gels are well known, and in fact many gels from various suppliers may be ordered according to the desired pore sizes and/or corresponding physical properties. In other cases, the pore size may be controlled by the gel maker during the gel-making process, by, for example. Controlling the crosslinking and/or bridging, determined by measuring the pores with light microscopy and/or determined by other techniques known in the art. In the present invention, the average pore size is greater than or equal to the average micelle diameter. In an embodiment of the invention, the average pore size is from about 1.05 times greater than the average micelle diameter to about 1000 times greater than the average micelle diameter. In an embodiment of the invention, the average pore size is from about 1.075 times greater than the average micelle diameter to about 10 times greater than the average micelle diameter.

In an optional step, a hydrotrope may be provided and added to the homogenizing step so as to reduce the average micelle diameter, provide easier processing, more uniform absorption of the liquid media, longer lasting absorption of the liquid media, and/or a more uniform gel appearance. Useful hydrotropes will depend greatly upon the actual liquid media and ingredient. In an embodiment herein the hydrotrope is a nonionic hydrotrope such as the Neodol® series from Shell Chemicals, Houston, Tex., USA; and/or various weights and variations of polyethylene glycol, commonly available in a variety of purities from industrial to food-grade from many companies worldwide. In an embodiment herein, the hydrotrope is a sulfonated hydrotrope, such as the alkali metal salts and alkali earth metal salts of xylene sulfonate, cumene sulfonate, and/or naphthalene sulfonate. In an embodiment herein, the hydrotrope is sodium cumene sulfonate. Surprisingly, it has been found that the addition of a carefully selected hydrotrope may also provide additional advantages, such as enhancing the odor impact of a perfume, and/or enhancing the absorption efficiency of the micelle into the gel.

The level of hydrotrope will vary greatly depending upon the actual ingredient and the liquid media. However, in an embodiment of the present invention, the hydrotrope is typically present at from about 0.01% to about 20% by weight of the mixture, preferably about 0.1% to about 10% by weight of the mixture, and more preferably from about 0.5% to about 5% by weight of the mixture.

A highly preferred ingredient in the present invention is a UV protector which is used herein to describe a material which absorbs, blocks and/or reflects UV light so as to reduce UV damage. Specifically, polymer molecules in the gel item and/or gel may degrade and/or break when exposed to light energy. Many light wavelengths, especially in the UV spectrum are known to affect polymer molecules by breaking and/or weakening the internal chemical bonds between monomers. In the case of gel items or gels, this may in some cases cause the shape of the gel item/gel to become deformed. In the case of gel items/gels which are formed into a specific regular shape, such as a block, a circle, a sphere, a star, etc., it may appear that the gel is melting over time. In an extreme case, the shape may be destroyed if excessive breaking of molecules occurring because of exposure to light during manufacture, shipping, storage, and/or use.

The possible detrimental effects of light are even stronger when a transparent or translucent package is used. In a highly preferred embodiment herein current product, a transparent package is used so that the regular shape of the gel item/gel is observable from the outside of the package.

Thus, useful UV protectors include the UV absorber SEESORB™ 101, available from Shipro Kasei Kaisha, Osaka, , Japan, which can be absorbed or otherwise incorporated into the gel. SEESORB™ 101 is a benzophenone based UV absorber. Also useful herein are benzo triazole based UV absorbers such as SEESORB 701, also available from Shipro.

Other examples of UV protectors which can be used alone or as a mixture with another UV protectors or with an anti-oxidant include the CYASORB UV series from American Cyanamid Co. (Wayne, N.J., USA) and the Tinogard TL series from Ciba Specialty Cehmicals Co. (Basel, Switzerland). Such UV protectors may be incorporated into any relevant portion of the product, for example, in to the packaging, into or onto the gel item, etc.

Anti-oxidants known in the art may also be useful herein to prevent degradation and/or damage to the gel item, perfume, and/or other ingredients in the product. While such anti-oxidants are well-known in the art, an example of a preferred anti-oxidant is SEENOX-BCS available from Shipro.

In order to improve UV, perfume, gel, and/or dye stability, it is preferred that the pH of any liquid component be from about 1.5 to about 5, preferably from about 2 to about 4, and more preferably from about 2.5 to about 3.5.

Other optional materials known in the art may be present as well, either in the mixture, gel item, or the process herein.

Test Methods:

The average pore size can be determined by analysis of the chemical structure of the gel and/or the gel item. In addition, certain gels and gel items may be ordered and/or designed to possess a certain pore size, shape, etc. As noted above, pore size may also be controlled by the gel maker during the gel making process, determined by taking measurements via light microscopy, and/or determined by other methods known in the art.

Micelle diameter is measured according to microscope analysis, or using a laser particle size measurement device.

Perfume impact is determined by a qualified perfume specialist and rated on a scale of 1 (not at all representative of the original perfume) to 10 (exactly the same as the original perfume).

Examples of the invention are set forth hereinafter by way of illustration and are not intended to be in any way limiting of the invention. The examples are not to be construed as limitations of the present invention since many variations thereof are possible without departing from its spirit and scope.

### EXAMPLE 1

3893.3 grams of deionized water is added in to a 5 liter tank connected to an IKA high shear mixer. After that, 62.5 grams of sodium cumene sulfonate (SCS), 455 grams of an odor-neutralizing polymeric active ingredient, 45.5 grams of phenoxyethanol, and 3.75 grams of dye solution are added into the tank. After that, the IKA high shear mixer is tuned on and run 5 minutes to homogenize the components to form a mixture. 11.8 g of a gel precursor in the form of dehydrated gel chips are placed in a flat pan. Within 10 minutes of forming the mixture, 118.2 mL of the mixture is poured into the pan. The pan is allowed to sit for 4 hours, resulting in a plurality of discrete gel units which have completely absorbed all of the mixture The average micelle diameter in the mixture is less than 5 μ, whereas the average pore size is about 10μ.

The perfume impact of the gel and the original perfume is identical as determined by a qualified perfume specialist. This example also gives an even perfume intensity over a two week period.

Comparative Example A is produced using the same process and materials, except that the high shear mixer is replaced with a paddle mixer. The average micelle diameter is significantly greater than 10 μ. The mixture is homogenized, but visible perfume droplets are noticed as the mixture is poured into the pan.

The perfume impact of the gel in Comparative Example A is noticeably different from that of the original perfume, as the top notes and bottom notes are separated, as determined by a qualified perfume specialist. Perfume oil is also seen coating the gel, and quickly pools in the bottom of the pan. Comparative Example A has a perfume intensity which is initially strong, but quickly decreases over 1 week.

As Comparative Example B, 2% di-propylene glycol is also added to the mixture of Example 1 which causes the average micelle diameter to increase to more than 10μ.

The perfume impact of the gel in Comparative Example B is noticeably different from that of the original perfume, as the top notes and bottom notes are separated, as determined by a qualified perfume specialist. Comparative Example B has a perfume intensity which decreases over time.

As Comparative Example C, the Example 1 is formed, except that no hydrotrope is added. The average micelle diameter is significantly greater than 10 μ. The mixture is homogenized, but visible perfume droplets are noticed as the mixture is poured into the pan.

The perfume impact of the gel in Comparative Example C is noticeably different from that of the original perfume, as the top notes and bottom notes are separated, as determined by a qualified perfume specialist.

### EXAMPLE 2

The gel of Example 1 is produced as described above as Example 2. for Comparative Example C, the hydrotrope is removed which causes the average micelle diameter to increase to more than 10μ.

The perfume impact of the gel in the comparative example is noticeably different from that of the original perfume, as the top notes and bottom notes are separated, as determined by a qualified perfume specialist. In addition, the perfume is noticeably on the outside of the gel, and in fact pools at the bottom of the tray.

All documents cited in the Detailed Description of the Invention are, are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A process for absorbing an ingredient into a gel item to form a gel comprising the steps of:
A. providing an ingredient and a liquid media, wherein the ingredient is insoluble in the liquid media;
B. homogenizing the ingredient and the liquid media in a mixer to form a mixture, the mixture comprising micelles suspended in the liquid media, wherein the micelles comprise the ingredient, and wherein the micelles have an average micelle diameter;
C. providing a gel item capable of absorbing the liquid media, the gel item having an average pore size, wherein the average pore size is greater than or equal to the average micelle diameter; and
D. absorbing the mixture into the gel item to form a gel.

2. The process of claim 1, wherein the liquid media is water.

3. The process of claim 1, wherein the average pore size is from about 1.05 times to about 1000 times greater than the average micelle diameter.

4. The process of claim 1, wherein the mixture further comprises a hydrotrope.

5. The process of claim 1, wherein the ingredient is a perfume.

6. The process of claim 1, wherein the mixer is a high shear mixer.

7. The process of claim 2, wherein the gel item is a dehydrated gel.

8. The process of claim 3, wherein the average pore size is from about 1.075 times to about 10 times greater than the average micelle diameter.

9. The process of claim 4, wherein the hydrotrope is a sulfonated hydrotrope

10. The process of claim 9, wherein the sulfonated hydrotrope is sodium cumene sulfonate.
